(19) United States
(12) Patent Application Publication
(10) Pub. No.: US 2021/0070628 A1
(43) Pub. Date: Mar. 11, 2021

(54) REMOVAL OF EUROPIUM IMPURITIES FROM SAMARIUM-153 IN NITRATE MEDIA USING IONIC LIQUIDS

(71) Applicant: SCK CEN (STUDIECENTRUM VOOR KERNENERGIE / CENTRE D'ETUDE DE L'ENERGIE NUCLEAIRE, Brussels (BE)

(72) Inventors: Koen BINNEMANS, Zele (BE); Thomas CARDINAELS, Mol (BE); Karen VAN HECKE, Mol (BE); Michiel VAN DE VOORDE, Heist-op-den-Berg (BE)

(21) Appl. No.: 16/756,937

(22) PCT Filed: Oct. 18, 2018
(86) PCT No.: PCT/EP2018/078528

§ 371 Date: Apr. 17, 2020

(30) Foreign Application Priority Data
Oct. 19, 2017 (GB) 1717166.1

(51) Int. Cl. C01F 17/10; C01F 17/20; A61K 33/244; C08F 212/08; C08F 212/36; B01D 15/36

(52) U.S. Cl. CPC C01F 17/10; C01F 17/20; A61K 33/244; C01P 2006/88; C08F 212/36; B01D 15/362; C08F 212/08

(57) ABSTRACT

A process of isolating samarium from a hydrophilic composition comprises nitrate ions, europium and samarium, by reducing europium(III) to europium(II) in this hydrophilic composition, and by extracting the samarium with a water-immiscible organic phase comprising an ionic liquid comprising nitrate anions.

### FIELD OF THE INVENTION

The present invention relates to a new solvent extraction based purification technique for the removal of europium impurities from a samarium oxide target for production of the samarium-153 medical radionuclide. The europium impurities are selectively reduced to their divalent oxidation state after dissolution of the irradiated material in nitrate media, whereafter the trivalent samarium is selectively extracted to the organic phase. Samarium can easily be stripped from the loaded organic phase by lowering the salt concentration in the system.

### BACKGROUND OF THE INVENTION

Like several other radiolanthanides, samarium-153 (¹⁵³Sm) can be applied in nuclear medicine, where it is known under its commercial names Quadramet® and Lexidronam®. These radiopharmaceuticals contain the ¹⁵³Sm radioisotope complexed to ethylene diamine tetra(methylene phosphonate) (¹⁵³Sm-EDTMP). ¹⁵³Sm serves well in nuclear medicine because of its favourable half-life of 46.284 h and because of its stable daughter isotope ¹⁵³Eu. This causes no severe side effects in the human body after treatment. The Quadramet® radiopharmaceutical is very selective towards the skeleton. Therefore, Quadramet® is frequently used in palliative treatment of painful osteoblastic bone metastases. These skeletal metastases are frequently caused by a variety of primary tumours, like lung cancer, prostate cancer, breast cancer or bone cancer. Quadramet® proves to be efficient in the treatment of these bone metastases because of the (β⁻-decay regime of ¹⁵³Sm. A pain reduction in over 70% of the patients was observed within a week after administration of Quadramet®, and had an average duration of more than 16 weeks. This way, the use of opioids can be largely reduced. Additionally, the treatment of these bone metastases can be monitored constantly by scintigraphy because of the simultaneous γ-emission of ¹⁵³Sm.

The ¹⁵³Sm radionuclide is commonly produced via neutron irradiation of an enriched ¹⁵²Sm₂O₃ target, using a high thermal neutron flux. This way, a product with high yield, high purity and high specific activity is formed. However, since the target is usually being irradiated for several days, part of the ¹⁵³Sm decays during its residence time in the nuclear reactor. Therefore, the daughter isotope ¹⁵³Eu will undergo neutron capture as well, leading to the production of trace amounts of ¹⁵⁴Eu. However, this radionuclidic impurity has a much longer half-life, i.e. 8.593 y, compared to ¹⁵³Sm, and would lead to an unacceptable radiation dose delivered to the patient. The maximum level of impurities are strictly regulated by international and national organizations (e.g. WHO, IAEA, FDA). Consequently, Quadramet® has a limited shelf life of only a few days after being produced, since expiration is reached at the threshold ratio of 0.093 μCi (=3400 Bq) of ¹⁵⁴Eu per mCi (37 MBq) of ¹⁵³Sm. Purification of the irradiated target could lead to a more efficient use of valuable target material and a higher availability of the radiopharmaceutical. Unfortunately, the separation of two neighbouring lanthanides is very challenging, because of their very similar chemical properties.

Since the 1950s, modern separation techniques such as ion exchange, solvent extraction and extraction chromatography have been further developed to obtain rare-earth compounds in high purity. To date, solvent extraction (SX) is the most important separation technique for rare earths on industrial scale. The separation of rare earths by solvent extraction depends upon the preferential distribution of rare earth complexes between two immiscible phases, typically an aqueous phase and an organic phase that are in contact with each other. However, a multistage counter-current separation method based on a very small difference in complexation behaviour would take too long time to be useful in the purification of these relatively short-lived radionuclides. Therefore, approaches based on the selective reduction of europium to its divalent state, both chemically and electrochemically, lead to a significant change in properties. Eu²⁺ has properties similar to the alkaline earth metal ions and an ionic radius that is very similar to that of Sr²⁺. This selective reduction approach already has proven to be efficient in methods developed for the recovery of highly valuable europium from rare-earth ores on industrial scale using highly acidic chloride media. In these techniques, a phosphor-based acidic extractant with high affinity for trivalent lanthanides (Ln³⁺), like bis(2-ethylhexyl)phosphoric acid (HDEHP, D2EHPA or P204), is used most frequently to extract all Ln³⁺ to an organic phase, leaving the Eu²⁺ unaffected in the chloride aqueous phase.

### SUMMARY OF THE INVENTION

This invention describes a method to separate europium impurities from the targeted ¹⁵³Sm isotopes to be used in radiopharmaceuticals. The method comprises a reduction step of europium(III) to europium(II) in nitrate aqueous solution to change its properties, followed by the actual separation step. The process allows for the separation of samarium-europium mixtures. The separation is achieved by the use of a water-immiscible nitrate ionic liquid without the use of any additional extractants. The process comprises a separation step, wherein the samarium-europium mixtures are separated by an extraction method using a selective extraction in two phases, whereby samarium is specifically extracted from a hydrophilic or aqueous phase to a water immiscible organic phase or ionic liquid phase, and wherein both phase comprise nitrate ions. In a specific embodiment, both phases do not contain Chloride ions and more specifically, both phases contain solely nitrate ions as anions.

The present invention is useful to separate europium from samarium, more specifically europium, including europium-154, is removed to purify samarium-153. Said purified samarium-153 is useful in medical applications and thus the present invention fulfils the need for more purified samarium-153, which contains less europium-154, so that the purified samarium-153 can be used more efficiently and for a longer period in said medical applications.

In a specific embodiment, the present invention relates to the purification process comprising following steps: (1) dissolution of the irradiated samarium oxide target material in nitric acid solution; (2) adjustment of the pH to a level between 4 and 7; (3) addition of a non-interfering nitrate salt to the aqueous solution to increase the total nitrate concentration in the aqueous feed solution to reach a very high ionic strength; (4) purging of the entire system with an inert gas to remove all dissolved oxygen; (5) reduction of europium to its divalent state; (6) contacting the aqueous feed solution with the organic phase comprising an ammonium based ionic liquid with nitrate anions to selectively extract the samarium ions; (7) separating the aqueous phase and the organic phase; (8) contacting the loading organic phase with water to reduce the total salt concentration of the system to strip the samarium from the loaded organic phase. Thus, the stripping solution in step (7) does not have to contain a water-soluble organic complexing agent (“complexant”). The separation is based on a different extraction behaviour of the lanthanide ions in different oxidation state. The extraction procedure in step (6) can also be performed with the ionic liquid immobilised on a solid support (i.e. with a supported ionic liquid phase, SILP). An important aspect of the invention is the reduction of europium in nitrate aqueous media. The very low concentration of the europium impurities might lead to insufficient stability of divalent europium as a function of time to perform the separation. The addition of stable europium isotopes increases the total europium concentration in the solution and therefore increases the stability of divalent europium over time.

The present invention relates to a process for separation a mixture of samarium and europium, comprising the solvent extraction of trivalent samarium ions from an aqueous phase comprising nitrate ions to a water-immiscible organic phase comprising an ionic liquid with nitrate anions, wherein the nitrate ions are able to form strongly extracted samarium(III) complexes, whereas no extractable europium(II) complexes are formed.

The concentration of the nitrate ions in the organic phase must be high. Therefore, the organic phase mainly consists of hydrophobic ionic liquids with nitrate ions. Ionic liquids are solvents that consist entirely of ions. The preferred ionic liquids are tricaprylmethylammonium nitrate (Aliquat 336) or benzyltrioctylammonium nitrate, but the invention is not restricted to these specific examples of ionic liquids. The ionic liquids have the general formulae [Q⁺][A⁻], where Q⁺ represents a cation and A⁻ represents an anion. Q⁺ has the general formula (R₁)(R₂)(R₃)(R₄)N⁺. R₁, R₂, R₃ and R₄ can be identical or different, and are a linear or branched alkyl chain, a cycloalkyl group, an aromatic group or a benzylic group, wherein said alkyl chain, cycloalkyl or aromatic groups are optionally substituted with one or more substituents selected from the group consisting of hydroxyl, amino, cyano, carboxyl, amide, ester, and nitro; and wherein said R₁, R₂, R₃, and/or R₄ optionally comprise at least one heteroatom, each of said heteroatoms being independently selected from the group consisting of nitrogen and oxygen. The total number of carbon atoms for R₁+R₂+R₃+R₄ is at least 24, preferably between 24 and 40. In an embodiment of the present invention, R₁, R₂, R₃ and R₄ can be a hydrogen atom, provided that the total number of carbon atoms for R₁+R₂+R₃+R₄ is at least 24. A⁻ is an anion capable of complexing lanthanide ions, preferably A⁻ is NO₃⁻. The ionic liquids [Q⁺][A⁻] can be used as pure compound or as mixtures of two or more [Q⁺][A⁻] compounds. In another embodiment of the present invention, the water-immiscible phase preferably fully complies with the CHNO principle, only containing carbon (C), hydrogen (H), nitrogen (N) and oxygen (O). This feature is highly important for the waste treatment of radioactively contaminated ionic liquids after use.

In addition to the ionic liquid, a fraction of the organic phase can be a diluent, or a diluent and a (phase) modifier can be added to change physicochemical properties such as solubility, viscosity, interfacial parameters, reducing adsorption losses and preventing third-phase formation. A diluent can be added to change the solubility and/or viscosity. A modifier or phase modifier can be added to change interfacial parameters, to reduce absorption losses and/or to prevent third-phase formation. Said diluents can be aliphatic hydrocarbons, aromatic hydrocarbons, ethers, alcohols or ketones, also fully complying with the CHNO-principle. The preferred diluents are aromatic hydrocarbons. Examples of such aromatic hydrocarbons include benzene, toluene, ethylbenzene, xylene, or commercial mixtures of aromatic solvents.

Typically, the aqueous feed solution contains lanthanide nitrates in concentrations between 0.1 and 2 g/L. Extra nitrate ions in the aqueous feed solution can be provided by the addition of non-interfering nitrate salts. Examples of such salts are ammonium nitrate, alkali metal nitrates and earth-alkaline nitrates. Selection of the non-interfering nitrate salt has to take the medical application of the purified samarium-153 product into account. The preferred pH is between 4 and 7. Lower pH values lead to a higher instability of europium(II), whereas higher pH values lead to hydrolysis of the lanthanide ions present in the aqueous solution.

The application of removal of europium impurities from the samarium-153 radionuclide by ionic liquids is not restricted to the use of conventional liquid-liquid extraction (solvent extraction processes), but it can be used in extraction chromatography as well. In extraction chromatography, the organic phase or ionic liquid phase is immobilised on a high-surface area solid support by impregnation to create supported ionic liquid phases (SILPs). The high-surface area solid support can be either organic (polymer) or inorganic (silica, alumina) based. Preferably, the solid support is a porous polystyrene-polydivinylbenzene copolymer or an insoluble aliphatic polyacrylic ester. The ionic liquid load on the solid support has to be limited to 1 g of ionic liquid per 1 g of solid support to prevent any ionic liquid losses to the aqueous phase during the purification procedure.

Another aspect of the present invention relates to a samarium composition, more specifically a samarium-153 composition, which contains no or less europium, more specifically europium-154, than what is present in the samarium-153 compositions of the prior art, more specifically in those samarium-153 compositions for medical applications, such as Quadramet® and Lexidronam®. Said samarium compositions or samarium-153 compositions are obtainable by the methods or processes of the present invention. Certain embodiments of the present inventions relates to the use of said samarium-153 compositions in medical applications, including treatment of cancer such as bone cancer, and as diagnostic use.

The present invention relates to a new solvent extraction process for the removal of long-lived europium-154 impurities from the irradiated ¹⁵²Sm₂O₃ target in the production of samarium-153 nuclides to be used in radiopharmaceuticals. After dissolution of the irradiated target material in nitrate environment, the europium impurities are reduced to their divalent oxidation state, changing their properties significantly. Samarium is extracted efficiently and selectively from an aqueous feed solution containing high concentration of nitrate ions to an organic phase. The organic phase comprises a water-immiscible ionic liquid with nitrate anions. The selected ionic liquid comprises a quaternised ammonium cation to fully comply with the CHNO principle. Moreover, the ionic liquid can be immobilised on an aromatic polymeric solid support to serve as a supported ionic liquid. Samarium can be easily stripped from the loaded organic phase by pure water or by reduction of the nitrate salt concentration in the aqueous phase.

Numbered statements of the invention are:
1. A process of isolating samarium from a hydrophilic composition comprising nitrate ions, europium and samarium, comprising the steps of:
   (a) reducing europium(III) to europium(II) in said hydrophilic composition; and
   (b) extracting said samarium with a water-immiscible organic phase comprising an ionic liquid comprising nitrate anions.
2. The process according to statement 1, wherein the samarium is ¹⁵³samarium.
3. The process according to statement 1 or 2, wherein the hydrophilic composition in step (a) does not contain chloride ions.
4. The process according to any one of statements 1 to 3, wherein the hydrophilic composition in step (a) and the water-immiscible organic phase in step (b) are consisting of nitrate ions as anions.
5. The process according to any of statements 1 to 4, further comprising step
   (c) back-extracting samarium to a hydrophilic composition wherein said hydrophilic solution has a lower concentration of nitrate ions than the nitrate ion concentration in step (a).
6. The process according to any one of statements 1 to 5, wherein the ionic liquid is tricaprylmethylammonium nitrate (Aliquat 336 nitrate) or benzyltrioctylammonium nitrate.
7. The process according to any one of statements 1 to 6, wherein the hydrophilic composition is an aqueous solution.
8. The process according to any one of statements 1 to 7, wherein the ionic liquid is impregnated on a solid support.
9. The process according to statement 8, wherein the solid support is a porous organic polymer.
10. The process according to statement 8 or 9, wherein the solid support is a porous polystyrene-polydivinylbenzene polymer or an insoluble aliphatic polyacrylic ester. The supported ionic liquid phase is for example the commercially available TEVA resin in its nitrate form.
11. The process according to statement 8, wherein the solid support is a porous inorganic support.
12. The process according to anyone of statements 1 to 11, wherein europium(III) is chemically, electrochemically (e.g. potentiostat), or photochemically reduced to europium(II) in step (a).
13. The process according to statement 12, wherein europium(III) is reduced to europium(II) by zinc powder.
14. The process according to any one of statements 1 to 13, wherein the stability of europium(II) is increased by the addition of stable europium isotopes in step (a).
15. The process according to any one of statements 1 to 13, wherein the stability of europium(II) is increased by the addition of a sacrificial agent in step (a).
16. The process according to any one of statements 1 to 15, wherein prior to step (a) the hydrophilic composition is purged with an inert gas.
17. The process according to any one of statements 1 to 16, wherein the pH of the hydrophilic composition is kept between pH4 and pH7.
18. The process according to any one of statements 8 to 17, wherein the supported ionic liquid phase is used in a column setup for separations via extraction chromatography.
19. The process according to any one of statements 1 to 18, wherein the nitrate concentration in the hydrophilic composition is at least 3 M.
20. The process according to any one of statements 1 to 19, wherein the nitrate concentration in the hydrophilic composition is about 6 M.
21. The process according to statement 19 or 20, wherein the nitrate concentration in the hydrophilic composition is obtained by addition of a salt selected from the group consisting of calcium nitrate, sodium nitrate, potassium nitrate, ammonium nitrate, lithium nitrate, and magnesium nitrate.
22. A composition comprising samarium-153, obtainable by the process of any of statements 1 to 21, characterised in that the amount europium-154 in said composition is less than 0.01% of the amount of samarium-153.
23. A composition comprising samarium-153 characterised in that the amount of europium-154 in said composition is less than 0.01% of the amount of samarium-153.
24. The composition according to statement 22 or 23, wherein said composition is free from europium-154.
25. A composition comprising samarium-153, obtainable by the process of any of claims 1 to 21, characterised in that the amount 154Europium in said composition is depleted by a factor of 4 or higher.
26. A composition comprising samarium-153 characterised in that the activity of europium-154 in said composition is less than 0.093 μCi per mCi of samarium-153.
27. The composition according to statement 25 or 26, wherein said composition is depleted in europium-154 and the expiration time of the said composition for radiopharmaceutical use is extended by several days compared to the undepleted composition.
28. A pharmaceutical composition, comprising the composition according to any of statements 22 to 27 and one or more pharmaceutically acceptable excipients.
29. The composition according to any of statements 22 to 27 for use as a medicament.
30. The composition according to any of statements 22 to 27 for use in the treatment of cancer.
31. The composition according to any of statements 22 to 27 for use in the treatment of bone cancer.
32. The composition according to any of statements 22 to 27 for use in a diagnostic method.
33. The use according to statement 32, wherein said diagnostic method is for the diagnosis and/or prognosis of a bone disease.
34. The use according to statement 33, wherein said bone disease is bone cancer.
35. The process according to any one of statement 1 to 22 further comprising the step of formulating the extracted samarium into a pharmaceutical composition.

### BRIEF DESCRIPTION OF THE DRAWINGS

FIG. 1: Change in effective magnetic moment, represented by the volume magnetic susceptibility, upon reduction of europium(III) to europium(II) as a function of time. The volume magnetic susceptibility was measured by means of a magnetic susceptibility balance. A solution containing approximately 10 g/L europium and 3 M Ca(NO₃)₂ was used. The reduction of europium(III) to europium(II) was performed chemically (dashed line, open circles) using zinc grains, and electrochemically (solid line, solid squares) by applying −0.7 V (vs. Ag/AgCl) using a reticulated vitreous carbon work electrode (WE) and a platinum wire counter electrode (CE). Both reduction experiments were performed at room temperature. Maximum reduction was reached after ca. 2 h for both reduction techniques.

FIG. 2: Distribution ratios as a function of time for a mixture of samarium(III) (squares) and europium(II) (circles) at approximately 1 g/L in a 6 M nitrate salt matrix (LiNO₃) in the aqueous feed solution when contacted with the organic phase based on tricaprylmethylammonium nitrate ([A336][NO₃], solid line, solid symbol) and benzyltrioctylammonium nitrate ([N_{b888}][NO₃], dashed line, open symbol). Samarium(III) is fully extracted. Extraction experiments are performed at 60° C. Europium was chemically reduced for at least 2 h prior to the extraction experiment.

FIG. 3: Separation factors as a function of time for a mixture of samarium(III) (squares) and europium(II) (circles) at approximately 1 g/L in a 6 M nitrate salt matrix (LiNO₃) in the aqueous feed solution when contacted with the organic phase based on tricaprylmethylammonium nitrate ([A336][NO₃], solid line, solid symbol) and benzyltrioctylammonium nitrate ([N_{b888}][NO₃], dashed line, open symbol). Tricaprylmethylammonium nitrate extracts more efficiently because of its lower viscosity. Extraction experiments are performed at 60° C. Europium was chemically reduced for a period of at least 2 h prior to the extraction experiment.

FIG. 4: Distribution ratios as a function of time for a solution of samarium(III) at approximately 1 g/L in the aqueous solution containing various nitrate salt concentrations (LiNO₃) when contacted with the organic phase based on tricaprylmethylammonium nitrate. Significant extraction rates for samarium(III) are reached at salt concentration of 3 M and higher. Extraction experiments are performed at 60° C. Europium was chemically reduced for a period of at least 2 h prior to the extraction experiment.

FIG. 5: Distribution ratios as a function of time at different extraction temperatures for a mixture of samarium(III) (squares) and europium(II) (circles) at approximately 1 g/L in a 6 M nitrate salt matrix (LiNO₃) in the aqueous feed solution when contacted with the organic phase based on tricaprylmethylammonium nitrate. No significant differences in extraction behaviour is observed when changing the temperature. Europium was chemically reduced for a period of at least 2 h prior to the extraction experiment.

FIG. 6: Samarium(III) and europium(II) are present in a concentration of approximately 1 g/L. Zinc(II) impurities are introduced after chemical reduction of europium(III) to europium(II), being present in a concentration of approximately 0.25 g/L. Zinc(II) is not extracted very efficiently to the organic phase. Europium was chemically reduced for a period of at least 2 h prior to the extraction experiment.

FIG. 7: Separation factors of Sm/Eu as a function of the mass supported ionic liquid phase used in extraction experiments using 2 mL of a feed solution containing a mixture of samarium(III) and europium(II) at approximately 1 g/L in a 6 M nitrate salt matrix (LiNO₃). Supported ionic liquid phases of tricaprylmethylammonium nitrate ([A336][NO₃]) and benzyltrioctylammonium nitrate ([Nb888][NO₃]) are compared with the neat solid support (polystyrene-polydivinylbenzene, Amberlite XAD-16N), using an ionic liquid loading of 1 gram per gram solid support. The aqueous solution and supported ionic liquid phase were contacted for 1 min at 25° C. The solid support itself proved to be inert over the entire series, whereas the supported ionic liquids proved to be able to separate samarium(III) and europium(II). It is clear that a higher amount of supported ionic liquid results in a better separation. The [A336][NO₃] containing supported ionic liquid results in the best separation. Europium was chemically reduced for a period of at least 2 h prior to the extraction experiment.

FIG. 8: Separation of samarium and europium using a commercially available supported ionic liquid phase (100-150 μm TEVA resin, consisting of tricaprylmethylammonium nitrate ([A336][NO₃]) immobilised on an insoluble aliphatic polyacrylic ester) as packing material in an extraction chromatography setup. A bed volume (BV) of 1.59 mL and a flow rate of 0.50 mL/min were used. An aliquot of 1.5 mL of a solution containing approximately 1 g/L samarium and approximately 0.5 g/L europium in 3 M Ca(NO₃)₂ was applied. Europium was chemically reduced for a period of at least 2 h prior to the extraction experiment. A blank 3 M Ca(NO₃)₂ solution was used as mobile phase. Demineralised water was used for stripping of samarium(III). All zinc(II) impurities and majority of europium(II) were eluted in the first fractions, whereas majority of samarium(III) was collected after stripping. Efficient separation can be achieved by proper fraction collection. In this example, a first fraction containing ca. 75 mass % europium and ca. 20 mass % samarium and a second fraction containing ca. 25 mass % europium and ca. 80 mass % samarium can be collected. Further optimisation of this column separation is still possible.

FIG. 9: Separation of samarium and europium using a commercially available supported ionic liquid phase (100-150 μm TEVA resin, consisting of tricaprylmethylammonium nitrate ([A336][NO₃]) immobilised on an insoluble aliphatic polyacrylic ester) as packing material in an extraction chromatography setup. A bed volume (BV) of 1.53 mL and a flow rate of 0.50 mL/min were used. An aliquot of 1.5 mL of a solution containing approximately 0.75 g/L samarium and approximately 0.75 g/L europium in 3 M Ca(NO₃)₂ was applied. Europium was electrochemically reduced for at least 2 h prior to the extraction experiment. A blank 3 M Ca(NO₃)₂ solution was used as mobile phase. Demineralised water was used for stripping of samarium(III). No zinc(II) impurities were present because of the electrochemical reduction technique. The majority of the europium was eluted in the first fractions, whereas all samarium(III) was collected in a concentrated fraction after stripping. Efficient separation can be achieved by proper fraction collection. In this example, a first fraction containing ca. 88 mass % europium and ca. 0 mass % samarium and a second fraction containing ca. 12 mass % europium and ca. 100 mass % samarium can be collected. Further optimisation of this column separation is still possible.

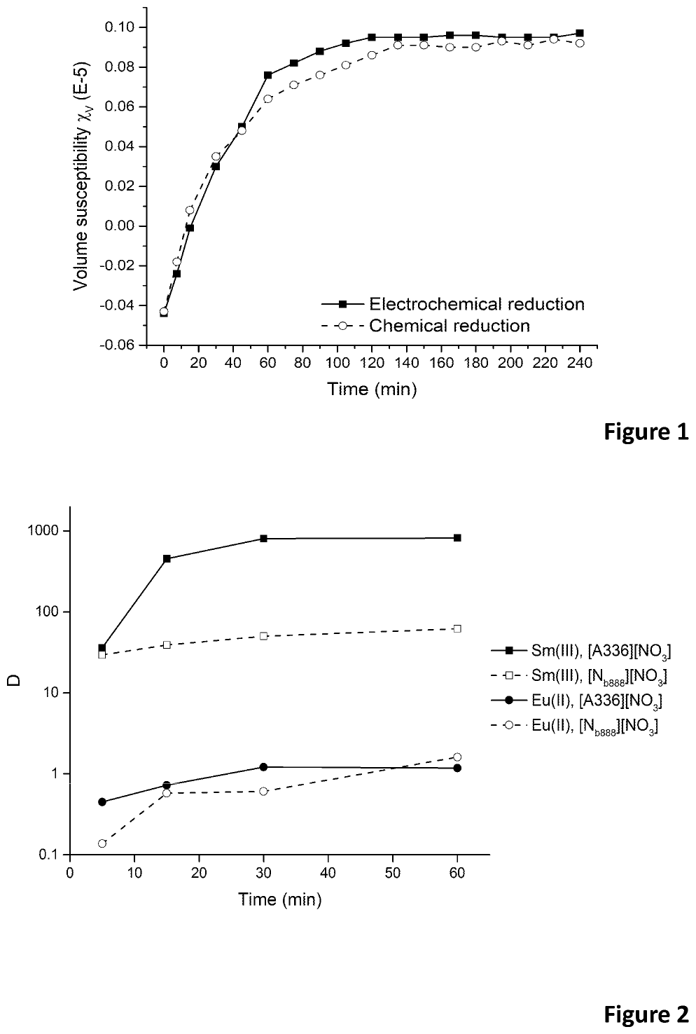

### DETAILED DESCRIPTION OF THE INVENTION

### Definitions

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” when referring to recited components, elements or method steps also include embodiments which “consist of” said recited components, elements or method steps.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

The recitation of numerical ranges by endpoints includes all numbers and fractions subsumed within the respective ranges, as well as the recited endpoints.

“Solvent extraction” refers to the distribution of a solute between two immiscible liquid phases in contact with each other, i.e. a two-phase distribution of a solute. In this invention, the solutes are the lanthanides samarium and europium, present in the form of a nitrate salt after dissolution of the oxide target material in a nitric acid solution. A lanthanide nitrate salt consists of a lanthanide cation and one or more nitrate anions. Solvent extraction, as applied to hydrometallurgy, is a unit operation for the purification and concentration of a wide variety of metals. In this invention, the metals of interest are samarium and europium. In general, solvent extraction comprises a step wherein an aqueous phase containing the metal of interest in contacted with an organic phase comprising an extractant. In the context of the present invention, said aqueous phase can be any hydrophilic composition that is immiscible with the organic phase. The extractant chemically reacts with the metal to form an organic-metal complex that is soluble in the organic phase. Impurities normally do not react with the extractant and remain in the aqueous phase. In this invention, the ionic liquid serves as both diluent and extractant. The organic phase, containing the organic-metal complex, is separated from the aqueous phase. The metal is recovered and concentrated into another aqueous phase by reversing the chemical reaction. Solvent extraction is often abbreviated to SX. “Liquid-liquid extraction” or “liquid-liquid distribution” are sometimes used as synonyms for solvent extraction.

“Extraction” is the operation of transferring the metal of interest from the aqueous of hydrophilic composition phase (“SX feed”, “aqueous feed” or “aqueous feed solution”) to the organic phase. The extraction circuit produces a loaded organic phase containing the metal value and an aqueous phase depleted of the metal known as raffinate. The raffinate is sent for further treatment or effluent.

“Stripping” (or “back extraction”) is the process of removing the metal of value from the organic phase by reversing the extraction chemical reaction. In the context of the present invention, the metal of value is samarium. It is normally conducted under conditions in order to produce a strip liquor containing a high concentration of the metal value. The strip liquor is the product of the solvent extraction circuit. General stripping refers to the (non-selective) stripping of the main solute(s) present, usually in one general stripping step. The term “selective stripping” is used where two or more main solutes are stripped successively from an extract, usually with different stripping agents with a view to their subsequent separate recovery from solution. “Regeneration” is the treatment of the stropped organic phase for removal of metals that were not scrubbed or stripped, or for the removal of organic degradation products. The operation produces a regenerated organic phase for recycle to the extraction operation as organic feed. The spent regenerant is advanced for further processing or to effluent treatment.

“Removing” europium impurities in context of the present invention refers to the selective transfer of samarium to the organic phase. Depending on the reaction conditions, samarium is after the extraction step for at least 50, 70, 75, 90, 95, 99, 99.5, 99.9, 99.99% present in the organic phase. Depending on the reaction conditions, when starting from a mixture of samarium and europium in the hydrophilic composition or aqueous phase, the organic phase can contain samarium and europium in different concentrations. The methods of the present invention accordingly allow to provide fractions that are enriched for samarium. This is typically achieved by batch wise or continuous repeatedly extractions. The methods of the present invention also allow to transfer in a single extraction step as much as possible of samarium to the organic phase. “Stripping” is the displacement of an impurity from an extract phase by contact with a solution containing the main extractable solute. The main solute need not be present in a pure solution but should have a higher distribution ratio than the impurities present. The “extract” is the separated phase (often but not necessarily organic) that contains the material extracted from the other phase. The “raffinate” is the phase remaining after extraction of some specified solute(s).

The “distribution ratio” (D) is the ratio of the total analytical concentration of a solute in the extract (regardless of its chemical form) to its total analytical concentration in the other phase. The “separation factor” is the ratio of the respective distribution ratios of two extractable solutes measured under the same conditions. The “loading” is the concentration of an extracted solute in the extract. The term “loaded solvent” is used to denote the extract after completion of a particular step, e.g. extraction or scrubbing. The “phase ratio” is the ratio of the quantity of the solvent to that of the other phase. Unless otherwise specified the phase ratio refers to the “phase volume ratio”.

A “sacrificial agent” is an agent that is added to the solution to react more favourable with interfering species to protect the species of interest, i.e. acting as a scavenger. For example, a species in its reduced oxidation state can be protected by adding an agent that is more prone to oxidation than the species of interest, thereby protecting the oxidation-sensitive species of interest.

A “diluent” is the liquid or homogeneous mixture of liquids in which extractant(s) and possible modifier(s) may be dissolved to form the solvent phase. Said diluent can be a molecular organic solvent (molecular diluent or organic molecular diluent) or an ionic organic solvent (ionic liquid diluent). Said molecular diluents can be aliphatic hydrocarbons, aromatic hydrocarbons, halogenated aliphatic hydrocarbons, halogenated aromatic hydrocarbons, ethers, alcohols or ketones. The preferred molecular diluents are aromatic hydrocarbons. Examples of such aromatic hydrocarbons/diluents include benzene, toluene, ethylbenzene or xylene. In this invention, quaternary ammonium or phosphonium salts with perchlorate anions are used as ionic liquid diluent. A “modifier” is a substance that can be added to the organic phase to change physicochemical properties such as solubility, interfacial parameters, reducing adsorption losses and preventing third-phase formation. The term “solvent” applies to the entire initial liquid phase containing the extractant. The solvent may contain only extractant or it may be a composite homogeneous mixture of extractant(s) with diluent(s) and also sometimes modifiers. In this context, the term “organic phase” is often used instead of “solvent”.

An “ionic liquid” is a solvent that consists entirely of ions (cations and anions). In this invention, ionic liquids are used as solvent for the extraction processes. An ionic liquid can act as a diluent, an extractant or as both extractant and diluent. A hydrophobic ionic liquid is an ionic liquid that phase-separates from an aqueous solution. The ionic liquid can be immobilised on a solid support in preparation of a “supported ionic liquid phase” (or SILP). The solid support is a porous network of inert solid material able to physically bind the ionic liquid after by means of impregnation. It is understood that the samarium and europium in the mixtures do not need to be present in equimolar concentrations. Samarium is present in large excess after irradiation of the enriched ¹⁵²Sm₂O₃ target. The exact concentration of samarium and europium is highly dependent on the irradiation parameters and the decay time between the end of the irradiation and the start of the purification process. Nevertheless, the total concentration of europium in the aqueous feed solution can be increased by the addition of stable europium isotopes (e.g. europium-153) to increase its stability in the divalent oxidation state. In certain embodiments of the present invention stable europium isotopes such as europium-153 are added to the hydrophilic composition or aqueous phase/solution. By said addition, the stability of europium(II) is increased in said hydrophilic composition or aqueous phase/solution.

In certain embodiments, the present invention relates to a process or a method of isolating samarium from a hydrophilic composition comprising nitrate ions, europium and samarium, comprising the steps of:
(a) reducing europium(III) to europium(II) in said hydrophilic composition; and
(b) extracting said samarium with a water-immiscible organic phase comprising an ionic liquid comprising nitrate anions.

In certain specific embodiments thereof, said samarium is samarium-153 (also described as ¹⁵³Sm throughout this invention).

In certain specific embodiments of the present invention, said hydrophilic composition does not contain chloride ions.

In more specific embodiments of the present invention, the hydrophilic composition and the water-immiscible organic phase in step (b) are consisting of nitrate ions as anions.

In certain embodiments, the present invention relates to a process or a method of isolating samarium from a hydrophilic composition comprising nitrate ions, europium and samarium, comprising the steps of:
(a) reducing europium(III) to europium(II) in said hydrophilic composition; and
(b) extracting said samarium with a water-immiscible organic phase comprising an ionic liquid comprising nitrate anions; and
(c) back-extracting samarium to a hydrophilic composition wherein said hydrophilic solution has a lower concentration of nitrate ions than the nitrate ion concentration in step (a).

In certain specific embodiments thereof, said samarium is samarium-153.

In certain specific embodiments of the present invention, the water-immiscible organic phase (used to extract samarium in step (b)) entirely consists of ionic liquid.

The ionic liquid as used in the present invention is preferentially tricaprylmethylammonium nitrate (Aliquat 336 nitrate) or benzyltrioctylammonium nitrate or a mixture of both ionic liquids.

In certain specific embodiments of the present invention, the hydrophilic composition is an aqueous solution or an aqueous phase which comprises nitrate ions and in more specific embodiments contains nitrate ions as anions.

In certain specific embodiments of the present invention, the ionic liquid is impregnated on a solid support. Said solid support can be a porous inorganic support or a porous organic polymer, such as a porous polystyrene-polydivinylbenzene polymer or an insoluble aliphatic polyacrylic ester.

In certain specific embodiments of the present invention, in step (a) europium(III) is chemically, electrochemically, or photochemically reduced to europium(II), and in certain specific embodiments thereof, extra europium or europium(III) is added in step (a). In more specific embodiments thereof, the reduction in step (a) is performed by adding zinc or zinc powder. Optionally, in step (a) a sacrificial agent can be added in order to increase the stability of europium(II) (also described as Eu(II) in this invention)

In certain specific embodiments of the present invention, prior to step (a) the hydrophilic composition is purged with an inert gas.

In certain specific embodiments of the present invention, the pH of the hydrophilic composition is kept between pH 4 and pH 7, in further specific embodiments said pH is about pH5 to about pH 6, or said pH is about 5.5.

In certain specific embodiments of the present invention, the supported ionic liquid phase is used in a column setup for separations via extraction chromatography.

In certain specific embodiments of the present invention, the nitrate concentration in the hydrophilic composition is at least 3 M, and in more specific embodiments thereof said nitrate concentration is between about 3 M and about 6 M; more specifically said nitrate concentration is about 6 M.

In certain specific embodiments of the present invention, the nitrate concentration in the hydrophilic composition is obtained by addition of a salt selected from the group consisting of calcium nitrate, sodium nitrate, potassium nitrate, ammonium nitrate, lithium nitrate, and magnesium nitrate.

Certain embodiments of the present invention relate to a composition comprising samarium-153, characterised in that the amount europium-154 in said composition is less than 0.1% or less than 0.01% or less than 0.001% of the amount of samarium-153. Said samarium-153 comprising composition is obtainable by the processes or methods of the present invention

Optimally said samarium-153 comprising composition is free from europium-154 or contains a residual europium-154 concentration, such that said samarium-153 comprising composition of the present invention can be used in medical applications in a more flexible way, i.e. the shelf life for the appropriate medical applications is longer than the shelf life/use with the presently available samarium-153 compositions, such as Quadramet® and Lexidronam®.

Certain embodiments of the present invention relate to said samarium-153 comprising compositions as pharmaceutical compositions comprising samarium-153 and one or more pharmaceutically acceptable excipients.

Certain embodiments of the present invention relate to the use of said samarium-153 comprising compositions and pharmaceutical compositions in medical applications, including the use as a medicament or a therapeutic treatment and further including the use as a diagnostic tool, such as the use in a diagnostic method for the diagnosis and/or prognosis of a bone disease. In further specific embodiments thereof, said use is for the treatment of cancer, such as bone cancer. In other specific embodiments, said bone disease is bone cancer, including bone cancer metastasis.

The invention applies to selectively reduce europium(III) to europium(II) to change its chemical properties prior to the separation step by extraction with an ionic liquid. The reduction can be performed by means of e.g. chemical (addition of zinc metal), electrochemical or photochemical techniques. This way the initial mixture of samarium and europium can be separated into the individual elements. In case of the ¹⁵²Sm₂O₃ target material, the aqueous feed solution is obtained by dissolution of the oxide in an aqueous solution of nitric acid. The pH of the aqueous solution is subsequently adjusted to enable reduction of europium(III) (4.5<pH<7), depending on the total europium concentration in solution. Highly acidic solutions should be avoided to prevent the oxidation of europium(II). Moreover, the aqueous feed solution is preferentially purged by an inert gas, like nitrogen gas or argon, to ensure the removal of any dissolved oxygen to prevent possible oxidation of europium(II). Oxidation of europium(II) causes the simultaneous extraction of europium(III) species with samarium(III) to the organic phase, leading to decreased separation efficiency. The extraction process is relatively independent of the pH, but the pH has to be sufficiently low to avoid hydrolysis of the dissolved lanthanide(III) ions. The extraction process is efficient at high nitrate concentrations, such as 6 M. Part of the nitrate ions can be provided by nitric acid used to dissolve the oxide target material, but in general extra nitrate salts have to be added to the aqueous feed solution. Thus, the aqueous feed solution is prepared for extraction by the addition of a non-interfering nitrate salt. Possible nitrate salts are e.g. ammonium nitrate (NH₄NO₃), alkali metal nitrates (LiNO₃, NaNO₃, KNO₃) or alkaline earth nitrates (Mg(NO₃)₂, Ca(NO₃)₂). Extra nitrate ions can also be provided by hydrogen nitrate (HNO₃). The acidity can be adjusted afterwards by addition of an oxide or hydroxide of an alkali metal or an alkaline earth metal or by the addition of an ammonia solution (NH_{3,aq}).

The concentration of nitrate ions in the organic phase must be as high as possible to increase the extraction capabilities. Therefore, hydrophobic ionic liquids with nitrate ions are selected as main components of the organic phase. Ionic liquids are solvents that consist entirely of ions. The ionic liquids have the general formulae [Q⁺][A⁻], where Q⁺ represents a cation and A⁻ represents an anion. Q⁺ has the general formulae (R₁)(R₂)(R₃)(R₄)N⁺. R₁, R₂, R₃ and R₄ can be identical or different, and are a linear or branched alkyl chain, a cycloalkyl group, an aromatic group or a benzylic group, wherein said alkyl chain, cycloalkyl or aromatic groups are optionally substituted with one or more substituents selected from the group consisting of hydroxyl, amino, cyano, carboxyl, amide, ester, and nitro; and wherein said R₁, R₂, R₃, and/or R₄ optionally comprise at least one heteroatom, each of said heteroatoms being independently selected from the group consisting of nitrogen and oxygen. The total number of carbon atoms for R₁+R₂+R₃+R₄ is at least 24, preferably between 24 and 40. In an embodiment of the present invention, R₁, R₂, R₃ and R₄ can be a hydrogen atom, provided that the total number of carbon atoms for R₁+R₂+R₃+R₄ is at least 24. A⁻ is an anion capable of complexing lanthanide ions, preferably A⁻ is NO₃⁻. The ionic liquids [Q⁺][A⁻] can be used as pure compound or as mixtures of two or more [Q⁺][A⁻] compounds, such as a mixture of tricaprylmethylammonium nitrate (Aliquat 336) and benzyltrioctylammonium nitrate. The ionic liquid can be used in a pure undiluted form or it can be mixed with a neutral extractant. The preferred ionic liquids are tricaprylmethylammonium nitrate (Aliquat 336) or benzyltrioctylammonium nitrate, but the invention is not restricted to these specific examples of ionic liquids. Other cations are substituted imidazolium, pyridinium, pyrrolidinium, morphoninium and piperidinium. Requirements are that the ionic liquids in combination with a nitrate anion phase-separates from the aqueous phase and has a very low solubility in water or preferably said ionic liquid is fully immiscible with water. Besides, the ionic liquid phase preferably fully complies with the CHNO-principle, only containing carbon (C), hydrogen (H), nitrogen (N) and oxygen (O). This feature is highly important for the waste treatment of radioactively contaminated ionic liquids after use. The dry ionic liquids [Q⁺][A⁻] often have a high viscosity. The viscosity can be lowered by using diluents, such as organic molecular diluents, by performing the extractions at elevated temperatures, between room temperature and 100° C., or by pre-saturation of the ionic liquid phase by an aqueous solution. Moreover, the addition of a diluent and a (phase) modifier can change other physicochemical properties such as solubility, interfacial parameters, reducing adsorption losses and preventing third-phase formation. Said diluents can be aliphatic hydrocarbons, aromatic hydrocarbons, ethers, alcohols or ketones, also fully complying with the CHON-principle. The preferred diluents are aromatic hydrocarbons. Examples of such aromatic hydrocarbons include benzene, toluene, ethylbenzene, xylene, or commercial mixtures of aromatic solvents. The application of ionic liquids is not restricted to conventional liquid-liquid extraction (solvent extraction processes). Ionic liquids can be used in extraction chromatography as well. In extraction chromatography, the organic phase or ionic liquid phase is immobilised on a high-surface area solid support by impregnation to create supported ionic liquid phases (SILPs). The high-surface area solid support can be either organic (polymer) or inorganic (silica, alumina) based. Preferably, the solid support is a porous polystyrene-polydivenylbenzene copolymer or an insoluble aliphatic polyacrylic ester. The ionic liquid load on the solid support is preferentially limited to 1 g of ionic liquid per 1 g of solid support to prevent any ionic liquid losses to the aqueous phase during the purification procedure.

The ratio of the volumes of the organic phase and the aqueous feed solution (phase ratio) can be varied. The volume of the organic phase should be sufficiently high in order to avoid exceeding the loading capacity of the organic phase. In cases where the organic phase consists only of an ionic liquid (and not added neutral extractant), the loading of the organic phase is preferably kept below 40 g/L. A diluent such as an organic molecular diluent in the organic phase should be used only if the viscosity of the organic phase is too high for convenient processing. The viscosity of the organic phase can be e.g. about 500 mPa·s in the processes of the present invention. The distribution ratios can be raised by addition of nitrate salts such as KNO₃, NaNO₃ or NH₄NO₃ to the aqueous phase, but this increases the amount of nitrate ions present in the aqueous phase.

Lanthanides(III), such as samarium(III), can be extracted from nitrate feed solutions by basic extractants in their nitrate form, i.e. hydrophobic ionic liquids with nitrate anions, provided that the ionic strength in the aqueous feed solution is sufficiently high, preferentially higher than 3 M. The lanthanides(III) are extracted as lanthanide(III) nitrates, rather than lanthanide(III) ions to the organic phase. Because the electrical neutrality is maintained during the extraction process, no ionic liquid components are lost in the aqueous phase due to extraction. In this sense, the extraction is similar to what is observed for extraction of lanthanides(III) by neutral extractants. The lanthanide(III) ions will be coordinated by nitrate ions in the ionic liquid phase to form anionic lanthanide(III) complexes. Nitrate ions not taking part in the coordination of lanthanide(III) ions serve as counter anion for the ionic liquid cations that are not involved as counter cations for the anionic lanthanide(III) complexes. Increase in the nitrate concentration leads to an increase in distribution ratio of lanthanide(III). It is assumed that the nitrate salts lower the activity of water, so that less water molecules are available for coordination to the lanthanide(III) ions, so that it is easier to dehydrate the lanthanide(III) ions upon extraction. The extraction process can be carried out by conventional solvent extraction equipment. After extraction, extracted metal ions with low distribution ratios, like europium(II) and zinc(II), can be scrubbed from the loaded organic phase by the addition of a fresh aqueous solution containing a high nitrate salt concentration, preferentially higher than 3 M. Divalent metal ions do not form extractable species in these media. A major advantage of this invention is that stripping of the lanthanide(III) ions from the loaded ionic liquid to an aqueous phase is easy, and that it does not require strong mineral acids. It is sufficient to bring the organic phase into contact with water to strip the lanthanide(III) ions to the aqueous phase. A lower concentration of nitrate salt (for instance 0.1 M ammonium nitrate) can be added to the stripping solution to facilitate phase separation of the organic and aqueous phase. By stripping of the lanthanide(III) ions from the ionic liquid phase by pure water, it is possible to obtain aqueous solutions of lanthanide(III) nitrates that are not contaminated by other metal nitrates. The lanthanide(III) can be recovered from the stripping solution, for example, by precipitation as oxalates with oxalic acid. Instead of adding oxalic acid to the aqueous stripping solution containing the lanthanide(III) ions, it is also possible to add solid oxalic acid to the loaded ionic liquid phase (precipitation stripping). In this way, the lanthanides(III) in the ionic liquid phase are transformed in one-step to the lanthanide(III) oxides. Calcination of the rare-earth oxalates leads to formation of rare-earth oxides. Pure chloride salts of samarium(III), as used in production of ¹⁵³Sm-EDTMP (Quadramet® or Lexidronam®), can be achieved by the addition of hydrochloric acid (HCl).

The ionic liquid can be pre-saturated with additional nitrate anions when the ionic liquids are prepared before contact with the aqueous feed solution in order to increase distribution ratios and loading capacity. This is done by contacting the ionic liquid with an aqueous solution containing a nitrate salt in high concentrations, preferentially higher than 3 M, which allows the ionic liquid to contain a slightly higher level of nitrate anions. This allows a greater deal of complexation for the extraction. This effect comes directly if the ionic liquid is prepared from for example a chloride form by contacting it with a concentrated aqueous solution of the corresponding desired ionic liquid anion. The ionic liquids used in the figures and examples are prepared in this pre-saturated form. This slightly increases the level of nitrate in the scrub or strip phases.

The ionic strength is an important parameter in these solvent extraction processes. First of all, the nitrate concentration has an influence on the distribution ratios during the initial solvent extraction process. The ionic strength also has an influence on the scrubbing and stripping steps. It is possible to change the distribution ratios by changing the concentration of the nitrate salt in the aqueous solutions. The aqueous phase can be sequentially contacted with different batches of the organic phase. In-between the different contacts the nitrate concentration in the aqueous phase is gradually increased or decreased. This approach is not restricted to the use of conventional liquid-liquid extraction (solvent extraction processes), but can be used in extraction chromatography as well. In extraction chromatography, the ionic liquid phase is immobilised on a high-surface area solid support by impregnation (supported ionic liquid phases or SILPs). The SILPs combine the advantages of ionic liquid solvent extraction systems and ion-exchange resins, and are very useful for the recovery of metals from diluted aqueous streams. Examples of solid supports are silica, alumina, activated carbon, or porous synthetic polymer resins such as polystyrene cross-linked by divinylbenzene or an insoluble aliphatic polyacrylic ester.

A person skilled in the art will understand that large variations in the composition of the aqueous feed solutions are possible, because the composition of the irradiated ¹⁵²Sm₂O₃ target is highly dependent on different irradiation parameters and the decay time allowed after irradiation. For instance the time spend in the nuclear reactor and the high thermal neutron flux used to irradiate the enriched ¹⁵²Sm₂O₃ target play a key role. Moreover, additional stable europium isotopes can be added for increased stability during reduction because it is more difficult to keep trace amounts of europium in their divalent state. In order to have the most efficient extraction, the extraction parameters need to be optimised for each different aqueous feed solution, a process within the normal skills of the skilled-in-the-art person. Depending on the starting material, process parameters that can be adjusted or optimised by the skilled person for an optimal process for said starting material are the ionic strength (salt concentration) of the feed solution, lanthanide(III) concentrations, the amount of reducing agent, the choice of reduction technique, the reduction time, the phase volume ratios (flow rates), the composition of organic phase (only basic extractants, or basic extractants combined with a diluent) and optimal temperatures for different stages.

The separation method can be used for the purification of irradiated ¹⁵²Sm₂O₃ target material to be used in radiopharmaceuticals. Nevertheless, the separation method can also be used for the removal of europium impurities from other rare earth elements in their trivalent oxidation state. Europium is the only rare-earth element that is sufficiently stable in the divalent state in aqueous media to be separated from the other rare earth elements in their trivalent oxidation state. Thus, the method of the present invention can also be used to separate other rare earth elements from europium. In non-nuclear applications, also other types of ionic liquids can be used, for example, phosphonium based ionic liquids, without the need to change the separation approach.

### EXAMPLES

### Example 1. Synthesis of Ionic Liquids

Tricaprylmethylammonium nitrate was prepared from the corresponding chloride ionic liquid by pre-equilibrating the chloride ionic liquid multiple times with a concentrated NH₄NO₃ solution to exchange the chloride ions for the nitrate ions. This resulted in water-saturated nitrate ionic liquids. Benzyltrioctylammonium nitrate was prepared by means of the quaternisation of trioctylamine by benzyl bromide while refluxing in acetonitrile for 8 h, resulting in the ionic liquid benzyltrioctylammonium bromide. The bromide anions where exchanged by nitrate anions by contacting the bromide ionic liquid with a concentrated NH₄NO₃ solution.

### Example 2. Chemical Reduction of Europium(III) by Zinc(0)

Europium(III) was reduced to europium(II) by contacting the aqueous feed solution with an excess of zinc metal grains (20 mesh) for over 2 h to assure high reduction rates. The pH of the solution was kept sufficiently high (4.5≤pH≤7) to prevent oxidation of europium(II) by H⁺. Moreover, the feed solution was purged by an inert gas (Ar or N₂) during the reduction in order to remove dissolved oxygen from the solution. It was observed that europium(II) is stable in aqueous media containing high nitrate salt concentrations. High nitrate concentrations (≥3 M) are needed to form stable europium(II)-nitrato complexes. Higher nitrate salt concentrations resulted in a higher stability. The formation of these europium(II)-nitrato complexes results in a change of colour of the solution, depending on the total europium concentration in the solution. The solution turns pale yellow, bright yellow or orange in case europium concentrations of 0.1, 1.0 or 10 g/L are present, respectively. The change in effective magnetic moment after reduction of europium(III), measured by means of a magnetic susceptibility balance, clearly showed the existence of europium(II) and its stability over time.

### Example 3. Electrochemical Reduction of Europium(III) Using a Potentiostat

Europium(III) was reduced to europium(II) in an electrochemical cell making use of a reticulated vitreous carbon (RVC) work electrode (WE) and a platinum (Pt) wire counter electrode (CE). A potential of −0.7 V versus a Ag/AgCl reference electrode (RE) was applied for the electrochemical reduction. The solution was purged by an inert gas (Ar or N₂) during the reduction to remove dissolved oxygen from the solution. The reduction was performed in a solution containing 10 g/L europium and 3 M Ca(NO₃)₂. The high nitrate salt concentration is needed to form stable europium(II)-nitrato complexes. The formation of these europium(II)-nitrato complexes resulted in a change of colour of the solution from colourless to orange. The change in effective magnetic moment after reduction of europium(III), measured by means of a magnetic susceptibility balance, clearly showed the existence of europium(II) and its stability over time.

### Example 4. Separation of Europium from Samarium Using Bulk Nitrate Ionic Liquids

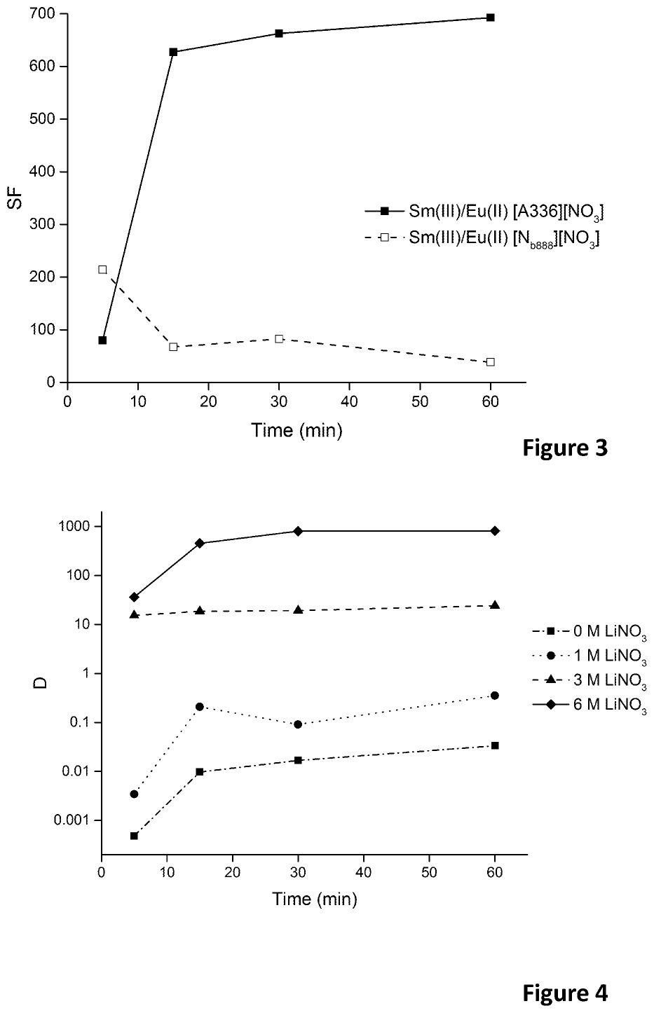

All separations between samarium and europium were determined between an aqueous solution with a 6 M nitrate matrix (6 M LiNO₃) containing both elements and a nitrate ionic liquid phase (tricaprylmethylammonium nitrate, benzyltrioctylammonium nitrate). The aqueous feed solutions contained approximately 1 g/L (0.0066 M) of each element. The extractions were carried out at 60° C. with a phase ratio of 1:1 (v/v). Both phases were mixed for different contact times, varying from 5 to 60 min, to find the lowest possible contact time. This way, less europium(II) can re-oxidize during separation, leading to worse separation rates. Besides, lower contact times result in faster separations, which is important in scope of the decaying samarium-153 nuclide over time. The obtained distribution ratios and separation factors for these experiments are shown in FIGS. 2 and 3, respectively.

It was found that both ionic liquids are able to extract the samarium(III) efficiently at high nitrate salt concentrations in a short period of time. Europium(II) was not extracted efficiently, leading to large separation factors. Higher separations can be achieved using tricaprylmethylammonium nitrate over benzyltrioctylammonium nitrate because of its lower viscosity. Lower viscosities improve the mass transfer.

### Example 5. Dependence on Nitrate Concentration for Tricaprylmethylammonium Nitrate Extractions

The nitrate matrix effect on extraction with tricaprylmethylammonium nitrate is shown in FIG. 4 by varying the concentration between 0 and 6 M nitrate (LiNO₃) on a solution containing approximately 1 g/L (0.0066 M) of samarium(III). The extractions were carried out at 60° C. with a phase ratio of 1:1 (v/v). Both phases were mixed for different contact times, varying from 5 to 60 min.

The conclusion to be drawn is that a high nitrate concentration is necessary to achieve an efficient extraction of samarium(III). Nitrate concentrations of 3 M or higher are preferred. Nevertheless, higher nitrate concentrations result in significantly higher distribution ratios for samarium(III), leading to a better and faster separation. Besides, sufficiently high nitrate concentrations are also preferred to form stable europium(II)-nitrato complexes in the aqueous solution, as mentioned in Example 2.

### Example 6. Dependence on the Temperature for Tricaprylmethylammonium Nitrate Extractions

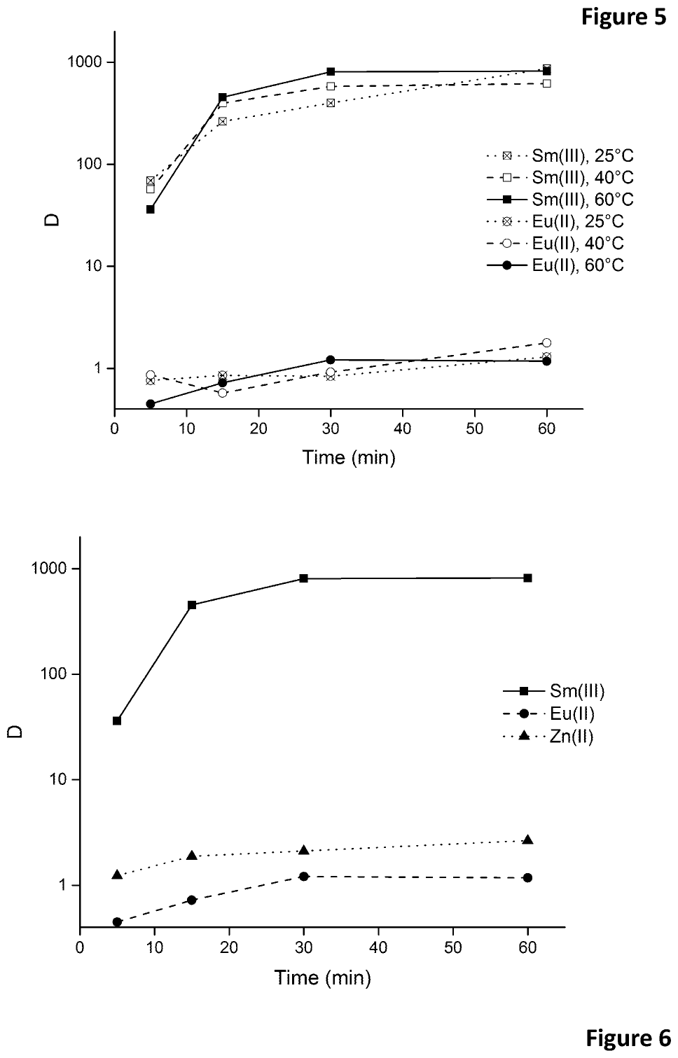

The dependence of the extraction of samarium(III) and europium(II) with tricaprylmethylammonium nitrate on temperature is shown in FIG. 5 for an aqueous solution with a 6 M nitrate matrix (6 M LiNO₃) containing both elements and the nitrate ionic liquid phase tricaprylmethylammonium nitrate. The aqueous feed solution contained approximately 1 g/L (0.0066 M) of each element. The extraction experiments were performed at different temperatures, varying from 25 to 40 and 60° C., with a phase ratio of 1:1 (v/v). For ionic liquids, a change in temperature leads to a significant reverse change in viscosity, effecting the mass transfer possibilities. Both phases were mixed for different contact times, varying from 5 to 60 min.

Extraction of samarium(III) proceeds slightly more efficient at elevated temperatures. Nevertheless, high distribution ratios are achieved for samarium(III) at every temperature tested, whereas the majority of europium(II) remains in the aqueous phase. Therefore, large separation factors can be achieved at every temperature tested.

### Example 7. Extraction Behaviour of Zinc(II), after Chemical Reduction of Europium(II), by Tricaprylmethylammonium Nitrate

The extraction behaviour of samarium(III), europium(II) and zinc(II) with tricaprylmethylammonium nitrate is shown in FIG. 6 as a function of time for an aqueous solution with a 6 M nitrate matrix (6 M LiNO₃) containing all three elements and the nitrate ionic liquid phase tricaprylmethylammonium nitrate. The aqueous feed solution contained approximately 1 g/L (0.0066 M) of both samarium(III) and europium(II). Zinc(II) is introduced in the aqueous feed solution after chemical reduction of europium(III) to europium(II) in the aqueous feed solution. On average, zinc(II) was present in a concentration of 0.25 g/L. The extractions were performed at 60° C. with a phase ratio of 1:1 (v/v)

Similarly to europium(II), zinc(II) is not well extracted to the ionic liquid phase in nitrate media. Therefore, zinc(II) impurities can be easily removed from the samarium(III) by means of a scrubbing step with a fresh aqueous solution containing high nitrate salt matrix.

### Example 8. Stripping of Tricaprylmethylammonium Nitrate

Stripping of tricaprylmethylammonium nitrate loaded with samarium(III) can be achieved very efficiently (over 99.5%) by reducing the nitrate salt concentration in the aqueous solution. In case stripping is performed with a pure water phase (0 M LiNO₃), samarium(III) can be fully recovered in an aqueous solution with a low salt concentration.

### Example 9. Immobilisation of Tricaprylmethylammonium Nitrate and Benzyltrioctylammonium Nitrate on a Polystyrene-Polydivinylbenzene Solid Support

Tricaprylmethylammonium nitrate and benzyltrioctylammonium nitrate were impregnated onto a polystyrene-polydivinylbenzene solid support for the production of the respective supported ionic liquid phases. A sample of 1 g of the ionic liquid was dissolved in acetone and subsequently shaken with 1 g of the dried and purified polymeric solid support material for 3 h to allow the ionic liquid to reach the inner pores of the polymeric solid support material. Afterwards, the acetone is slowly evaporated in a rotary evaporator to assure a homogeneous distribution of the ionic liquid film over the polymeric solid support particles. The supported ionic liquid phases are allowed to dry in high vacuum to remove remaining acetone traces.

### Example 10. Separation of Samarium and Europium Using Supported Ionic Liquid Phases

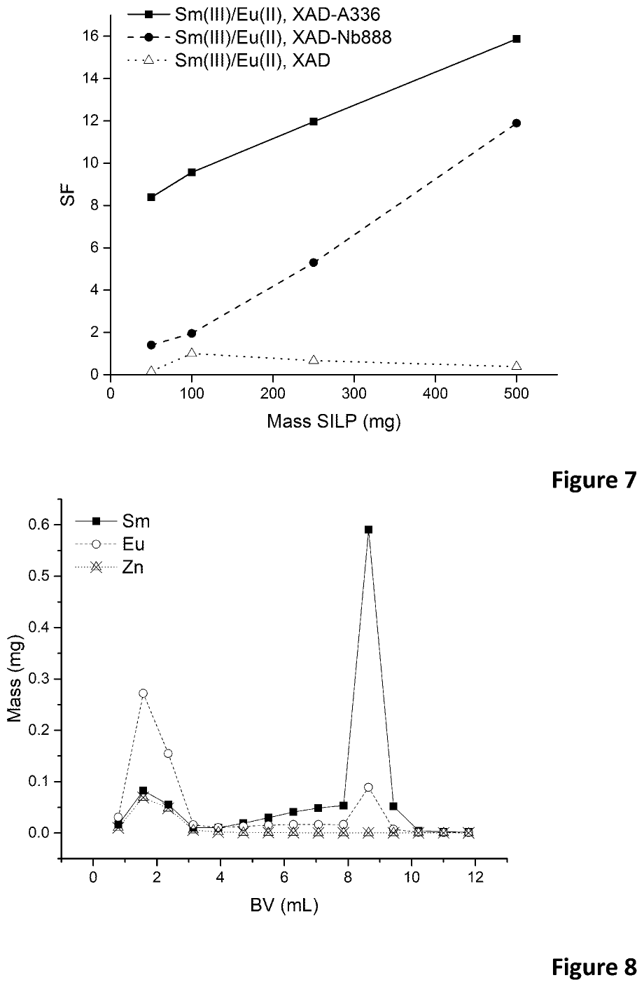

The supported ionic liquid phases containing tricaprylmethylammonium nitrate and benzyltrioctylammonium nitrate were used in batch extraction experiments. Their performance was compared to the use of neat polystyrene-polydivinylbenzene to prove the inertness of the solid support used for their preparation. A sample of 50, 100, 250 and 500 g solid material was contacted with 2 mL of an aqueous feed solution containing 6 M LiNO₃ and approximately 1 g/L (0.0066 M) of both samarium(III) and europium(II). Results of these extractions are shown in FIG. 7 by means of the fraction extracted for a contact time of 1 minute. The temperature during these experiments was kept constant at 25° C.

Similar to the bulk ionic liquids, samarium(III) is extracted well to the supported ionic liquid phase, whereas europium(II) and zinc(II) remain in the aqueous phase. The efficiency of the separations increases with increasing amount of supported ionic liquid. Moreover, it is clear that the supported ionic liquid phase based on tricaprylmethylammonium nitrate is most efficient in separating samarium(III) and europium(II).

### Example 11. Separation of Samarium and Europium Using Supported Ionic Liquid Phases in Extraction Chromatography

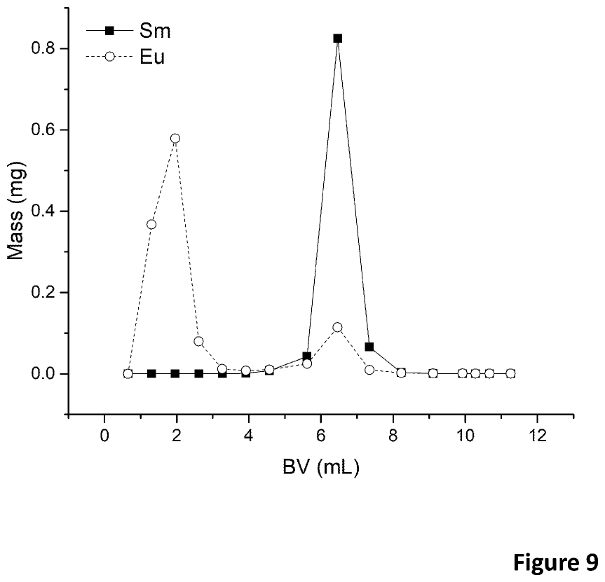

A commercially available supported ionic liquid phase, consisting of tricaprylmethylammonium nitrate immobilised on an insoluble aliphatic polyacrylic ester (i.e. TEVA resin, 100 to 150 μm), was used in extraction chromatography experiments. A bed volume of approximately 1.6 mL was used. After (electro-)chemical reduction of europium, an aliquot of 1.5 mL containing 1 g/L Sm and 0.1 to 1 g/L Eu in a 3 M Ca(NO₃)₂ solution was loaded onto the column. The column was eluted using a blank 3 M Ca(NO₃)₂ solution. Europium(II) and zinc(II) originating from the chemical reduction step were not retained, whereas samarium(III) and any remaining europium(III) were extracted to the thin ionic liquid layer of the supported ionic liquid phase. Zinc(II) impurities are not present if an electrochemical reduction technique is applied for the reduction of europium(III) to europium(II). Plain water was used as a stripping agent as reduction of the salt concentration in the system ensures an efficient back-extraction of samarium(III) to the aqueous phase. FIG. 8 demonstrates the feasibility to separate europium(II) and zinc(II) from samarium(III) after a 2 h chemical reduction of europium(III) to europium(II). FIG. 9 demonstrates the separation of europium(II) from samarium(III) after a 2 h electrochemical reduction of europium(III) to europium(II), without the introduction of any zinc impurities. 75 to 90% europium can be removed in a single run. Further optimisation of this extraction chromatography technique is still possible.

### Example 12. Extension of the Expiration Date of the Samarium-153 Radioisotope for Radiopharmaceutical Use

Samarium-153 is most efficiently produced by irradiating an enriched samarium-152 target using a high thermal neutron flux in a nuclear research reactor. If a target is irradiated by a high thermal neutron flux of 3.5×10¹⁴ n cm's⁻¹, a theoretical activity of 5650 Ci/g ¹⁵²Sm can be reached for samarium-153 after 4 days of irradiation. The activity of europium-154 in this irradiated target would be 0.040 Ci/g ¹⁵²Sm. A maximum activity of 6900 Ci/g ¹⁵²Sm for samarium-153 can be reached after 9 days of irradiation, with a europium-154 activity of 0.300 Ci/g ¹⁵²Sm. The threshold value for long-lived europium-154 impurities for samarium-153 radiopharmaceuticals has been established at 0.093 μCi/mCi ¹⁵³Sm by regulating agencies. This threshold value would be reached after 7 days when irradiating for 4 days, leaving 435 Ci/g ¹⁵²Sm untouched. After 9 days of irradiation, the threshold value would already be reached after 2 days, leaving 3240 Ci/g ¹⁵²Sm untouched and making processing and distribution impossible. If the irradiated target material is depleted in europium-154 by a factor of five using a radiochemical purification step, the time to expiration can be extended by 4.5 days. After 4 days of irradiation, the samarium-153 radiopharmaceutical can be used up to 11.5 days, leaving only 72 Ci/g ¹⁵²Sm untouched. The samarium-153 radiopharmaceutical can be used up to 6.5 days after 9 days of irradiation, with a remaining 540 Ci/g ¹⁵²Sm. Higher depletion factors can lead to even higher extension of the expiration date.

1.-23. (canceled)

24. A process of isolating samarium from a hydrophilic composition comprising nitrate ions, europium and samarium, the process comprising the steps of:
a) reducing europium(III) to europium(II) in said hydrophilic composition; and
b) extracting said samarium with a water-immiscible organic phase comprising an ionic liquid comprising nitrate anions.

25. The process according to claim 24, wherein said samarium is samarium-153.

26. The process according to claim 24, wherein the hydrophilic composition in step a) does not contain chloride ions.

27. The process according to claim 24, wherein the anions in the hydrophilic composition in step a) and in the water-immiscible organic phase in step b) consist of nitrate anions.

28. The process according to claim 24, further comprising the step of:
c) back-extracting samarium to a hydrophilic composition wherein said concentration of nitrate ions is lower than the concentration of nitrate ions in the hydrophilic composition of step a).

29. The process according to claim 24, wherein the ionic liquid is tricaprylmethylammonium nitrate or benzyl¬trioctyl¬ammonium nitrate.

30. The process according to claim 24, wherein the hydrophilic composition is an aqueous solution.

31. The process according to claim 24, wherein the ionic liquid is impregnated on a solid support.

32. The process according to claim 31, wherein the solid support is a porous organic polymer.

33. The process according to claim 32, wherein the porous organic polymer is a polystyrene-polydivinylbenzene copolymer, or a insoluble aliphatic polyacrylic ester.

34. The process according to claim 31, wherein the solid support is a porous inorganic support.

35. The process according to claim 24, wherein europium(III) is chemically, electrochemically, or photochemically reduced to europium(II) in step a) and/or wherein europium(III) is chemically reduced to europium(II) by zinc powder or wherein europium(III) is electrochemically reduced to europium(II) with a potentiostat.

36. The process according to claim 24, wherein the stability of Eu(II) is increased by the addition of stable europium isotopes in step (a).

37. The process according to claim 24, wherein the stability of Eu(II) is increased by the addition of a sacrificial agent in step (a).

38. The process according to claim 24, wherein prior to step (a) the hydrophilic composition is purged with an inert gas.

39. The process according to claim 24, wherein the pH of the hydrophilic composition is kept between pH 4 and pH 7.

40. The process according to claim 31, wherein samarium is extracted by chromatography on a column comprising said impregnated solid support.

41. The process according to claim 24, wherein the nitrate concentration in the hydrophilic composition is at least 3 M or wherein the nitrate concentration in the hydrophilic composition is about 6M.

42. The process according to claim 41, wherein nitrate in the hydrophilic composition is added as a salt selected from the group consisting of calcium nitrate, sodium nitrate, potassium nitrate, ammonium nitrate, lithium nitrate, and magnesium nitrate.

43. The process according to claim 24 further comprising the step of formulating the extracted samarium into a pharmaceutical composition.
